(19) United States
(12) Patent Application Publication
(10) Pub. No.: US 2025/0011297 A1
(43) Pub. Date: Jan. 9, 2025

(54) PROCESS AND INTERMEDIATES FOR PREPARATION OF ISOFETAMID

(71) Applicant: ADAMA MAKHTESHIM LTD., Beer Sheva (IL)

(72) Inventors: Michael GRABARNICK, Metar (IL); Wei LIU, Beijing (CN); Shaoxiang WU, Beijing (CN); Rui GAO, Beijing (CN); Rui WANG, Beijing (CN); Wenjun WANG, Beijing (CN)

(73) Assignee: ADAMA MAKHTESHIM LTD., Beer Sheva (IL)

(21) Appl. No.: 18/709,372

(22) PCT Filed: Nov. 11, 2021
(86) PCT No.: PCT/CN2021/130096

§ 371 Date: May 10, 2024

(51) Int. Cl. C07D 333/38; C07C 41/09; C07C 45/42; C07C 249/02; C07C 251/24; C07F 3/02

(52) U.S. Cl. CPC C07D 333/38; C07C 41/09; C07C 45/42; C07C 249/02; C07C 251/24; C07F 3/02

(57) ABSTRACT

The present disclosure relates to novel intermediates, preparation process thereof, and a process for producing isofetamid. The present disclosure also relates to the use of novel intermediates for preparing isofetamid. The present disclosure further relates to a process for preparation of some other intermediates of isofetamid using the novel intermediates.

### TECHNICAL FIELD OF THE INVENTION

The present disclosure relates to novel intermediates, preparation process thereof, and a process for producing isofetamid. The present disclosure also relates to the use of the novel intermediates for preparing isofetamid. The present disclosure further relates to a process for preparation of some other intermediates of isofetamid using the novel intermediates.

### FIELD AND BACKGROUND OF THE INVENTION

Isofetamid, having the chemical name N-[1,1-dimethyl-2-(4-isopropoxy-o-tolyl)-2-oxoethyl]-3-methylthiophene-2-carboxamide, has the structural formula:

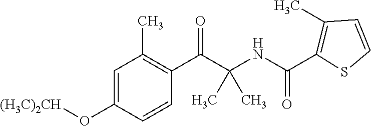

Isofetamid is a phenyl-oxo-ethyl thiophene amide fungicide. Isofetamid is a broad-spectrum fungicide belonging to the Succinate Dehydrogenase Inhibitors (SDHI) group. It inhibits succinate dehydrogenase in complex II of fungal mitochondrial respiration and is used to control fungal pathogens belonging to Ascomycetes pathogens such as Monilinia spp., Sclerotinia spp. and Deuteromycetes pathogens, such as Botrytis spp. It has efficacy in each stage of the biological cycle of the fungus i.e. spore germination, germ tube growth, penetration, mycelial growth and sporulation. Isofetamid has translaminar properties.

Isofetamid was disclosed by Ishihara Sangyio Kaisha, Ltd. in PCT patent applications WO 2003/027059 and WO 2006/016708. Both WO2003/027059 and WO 2006/016708 describe a method of preparing isofetamid.

Processes for producing intermediates of isofetamid were described in CN 102503751, WO 2018/197324, CN 101928208, CN 109534976 and CN 111548257.

CN 102503751 discloses a method for producing an alpha-brominated aromatic ketones compound. The method comprises taking an aromatic ketones compound as a substrate, hydrogen bromide as a brominating agent, copper nitrate as a catalyst, oxygen or air as an oxidizing agent and water as a solvent. WO 2018/197324 discloses a process for reacting an alkyl aryl ketone obtaining thereby the corresponding aryl oxirane or α-functionalized alkyl aryl ketal, the aryl oxirane or α-functionalized alkyl aryl ketal obtained by the process as well as the α-functionalized ketone obtained by the process.

CN 101928208 discloses a process for synthesizing an α-brominated ketone compound by oxidation-bromination with hydrogen peroxide.

CN 109534976 discloses a method for preparing α-hydroxy ketone in the presence of an acyl chloride and hexafluoroisopropanol.

CN 111548257 discloses a method for producing (4-isopropoxy-2-methyl) phenyl isopropyl ketone (compound of Formula I).

The development of one-pot syntheses, in which at least two sequential transformations are performed in a single reaction flask, has recently gained considerable attention. This interest is due to the increasing concern about sustainable chemistry since it is related with saving resources and with the reduction of the produced waste compared with the traditional processes. Generally, after each chemical transformation the process is stopped previous to the subsequent reaction pathway in order to eliminate the reaction media and/or for the purification and isolation of the reaction intermediate. In this context, at industrial scale, one-pot approach could be the best solution to reduce time, costs, resources and waste generation, since these processes would avoid the purification of the intermediates between individual steps, where major efforts are invested. Moreover, by reducing the number of synthetic steps and avoiding the purification processes, it is possible to reduce the loss of material and thus to increase the overall yield of the reaction. Therefore, one-pot process is hugely attractive for the synthesis of active compounds.

Novel substituted imines of formula (V) described as follows are not reported in the literature. Said substituted imines are useful chemical intermediates which are prepared from commercially available raw materials in high yields and good quality in an economically advantageous and easily handled way.

The present disclosure discloses novel intermediates for producing isofetamid as well as process for producing the key intermediates of isofetamid. In this invention, one-pot synthesis of the key intermediates and telescopic process for preparing the key intermediates are also disclosed.

It would be highly desirable to have an improved process for the production of the compound of formula (I) described as follows which is suitable for industrial use, highly efficient, low-cost, environmentally friendly, and provides a high yield in a relatively short reaction time, thereby overcoming the deficiencies of the prior art. The present subject matter provides such a process.

### SUMMARY OF THE INVENTION

The present disclosure relates to novel intermediates, preparation process thereof, and a process for producing isofetamid. The present disclosure also relates to the use of the novel intermediates for preparing isofetamid. The present disclosure further relates to a process for preparation some other intermediates of isofetamid using the novel intermediates.

Specifically, the present disclosure provides the following embodiments:

1. A compound of Formula (V)

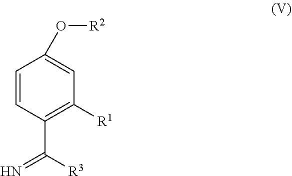

wherein:

R¹ is H, a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl;

R² is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and

R³ is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl.

2. The compound of embodiment 1, wherein R¹ is H, a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R² is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R³ is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl.

3. The compound of embodiment 1, wherein R¹ is H, a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R² is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R³ is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl.

4. The compound of embodiment 1, wherein R¹ is H, a C₁-C₃ alkyl, or a C₃-C₆ cycloalkyl, in which the alkyl may be substituted with a halogen, and the cycloalkyl may be substituted with a halogen, a C₁-C₆ alkyl or haloalkyl; R² is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl; wherein R³ is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl.

5. The compound of embodiment 1, wherein R¹ is H or a C₁-C₃ alkyl, in which the alkyl may be substituted with a halogen; R² is a C₁-C₄ straight or C₃-C₄ branched alkyl, in which the alkyl may be substituted with a halogen; R³ is a C₁-C₄ straight or C₃-C₄ branched alkyl, in which the alkyl may be substituted with a halogen.

6. The compound of embodiment 1, wherein R¹ is C₁-C₃ straight alkyl, in which the alkyl may be substituted with a halogen; R² is a C₃-C₄ branched alkyl, in which the alkyl may be substituted with a halogen; R³ is a C₃-C₄ branched alkyl, in which the alkyl may be substituted with a halogen.

7. The compound of embodiment 1, wherein R¹ is methyl; R² is isopropyl; and R³ is isopropyl.

8. A process for preparing a compound of Formula (V) according to any one of embodiments 1 to 7,

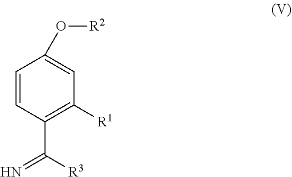

comprising:
b) reacting a compound of Formula (III) with a compound of Formula (IV)

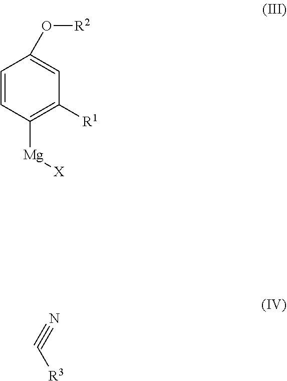

or
b1) reacting a compound of Formula (XII) with a compound of Formula (XIII)

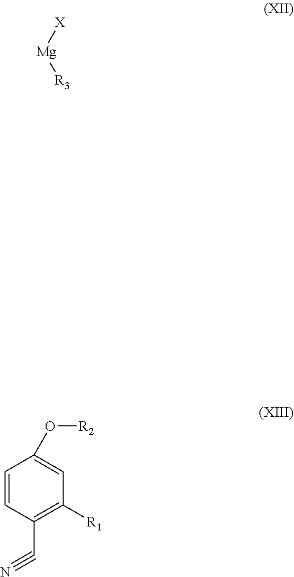

to prepare a compound of Formula (V);

wherein:

X is a halogen;

R¹, R², and R³ are defined as any one of embodiments 1 to 7.

9. The process of embodiment 8, wherein when the process comprises a step b), the process further comprises a step a) reacting the compound of Formula (II) with magnesium to prepare the compound of Formula (III)

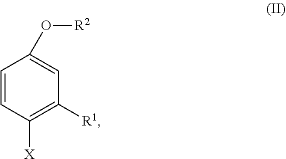

wherein: X, R¹, and R² are defined as in embodiment 8.

10. The process of embodiment 8, wherein when the process comprises a step b1), the process further comprises a step al) reacting the compound of Formula (XI) with magnesium to prepare the compound of Formula (XII)

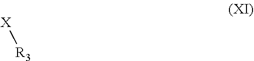

wherein: X and R³ are defined as in embodiment 8.

11. The process of embodiment 9, wherein steps a) and b) are carried out sequentially as a telescopic process.

12. The process of embodiment 10, wherein steps al) and b1) are carried out sequentially as a telescopic process.

13. The process of any one of embodiments 9 and 11, wherein the process further comprises:
d) reacting the compound of Formula (VI) with an alkylating agent

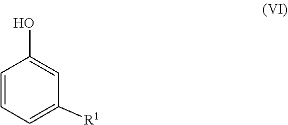

to prepare a compound of Formula (VII)

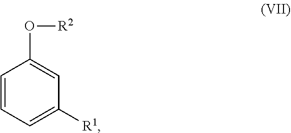

e) halogenating the compound of Formula (VII) with a halogenating agent to prepare the compound of Formula (II),

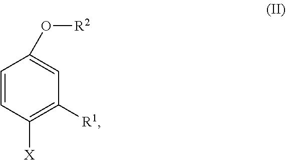

wherein, X, R¹ and R² are defined as in embodiment 9.

14. The process of embodiment 13, wherein step d) is carried out with an alkyl halide and in the presence of a base.

15. The process of embodiment 13, wherein step e) and step d) are carried out sequentially as a telescopic process.

16. The process of embodiment 14, wherein the base is selected from the group consisting of alkali metal hydroxides, alkali metal carbonates, hydrides, alkaline earth metal hydroxides and alkaline earth metal carbonates.

17. The process of embodiment 13, wherein the halogenating agent in step e) is a chlorinating agent selected from the group consisting of NCS, Cl₂, dichlorodimethyl hydantoin, trichloroisocyanuric acid, N-chlorophthalimide, sulfuryl chloride and the mixtures thereof.

18. The process of embodiment 13, wherein the halogenating agent in step e) is a brominating agent selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid, bromodimethylsulfonium bromide, 5,5-dibromomeldrum's acid, bis(2,4,6-trimethylpyridine)-bromonium hexafluorophosphate, bromine monochloride and the mixtures thereof.

19. The process of any one of embodiments 13-18, wherein the halogenating in step e) is carried out by oxyhalogenating process using an oxidizing agent and source of halogen ions under acidic conditions.

20. The process of embodiment 19, wherein the oxidizing agent is selected from the group consisting of hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, oxone, DMSO and mixtures thereof.

21. The process of any one of embodiments 19-20, wherein the source of halogen ions is a hydrogen halide or a mixture of a strong acid and an alkali metal or alkaline earth metal salt of a hydrogen halide.

22. The process of any one of embodiments 13-21, wherein a phase transfer catalyst is used in step d) and/or step e).

23. The process of any one of embodiments 8-22, wherein X is Cl or Br.

24. A process for preparing a compound of Formula (I),

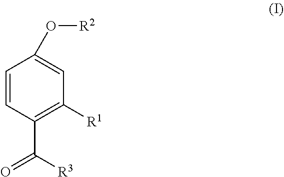

comprising:
B) preparing the compound of Formula (V) according to the process of any one of embodiments 8 to 23;

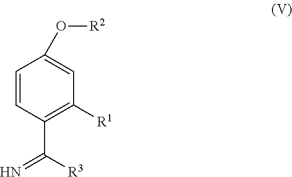

c) hydrolyzing the resulting compound of Formula (V);

wherein: R¹, R², and R³ are defined as any one of embodiments 8 to 23.

25. The process of embodiment 24, wherein the hydrolysis in step c) is acid-catalyzed hydrolysis or base-catalyzed hydrolysis.

26. The process of any one of embodiments 24-25, wherein when the process comprises steps a), b) and c), steps a) and b), steps b) and c), or steps a), b) and c) are carried out sequentially as a telescopic process.

27. The process of any one of embodiments 24-25, wherein when the process comprises steps al), b1) and c), steps al) and b1), steps b1) and c), or steps al), b1) and c) are carried out sequentially as a telescopic process.

28. A process for preparing a compound of Formula (X) comprises:
i) halogenating a compound of Formula (I) with a halogenating agent to prepare a compound of Formula (VIII)

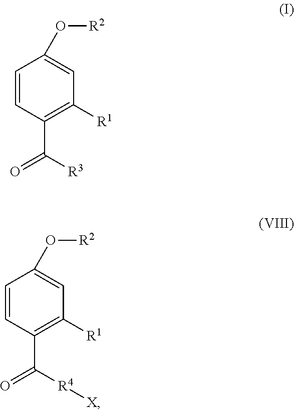

ii) substitution in the compound of Formula (VIII) to prepare a compound of Formula (IX)

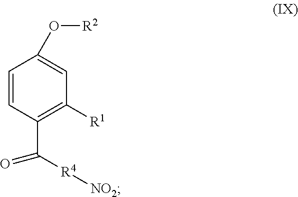

iii) reducing the compound of Formula (IX) to prepare a compound of Formula (X)

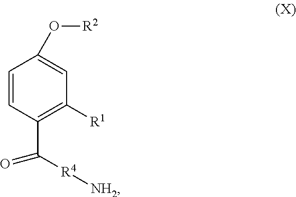

wherein:

X is a halogen;

R¹ is H, a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl;

R² is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and

R³ is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and

R⁴ is a C₁-C₁₀ straight or C₃-C₁₀ branched alkanediyl, a C₃-C₁₀ cycloalkylene, a C₇-C₁₀ aralkylene, or a C₆-C₁₀ arylene, in which the alkanediyl may be substituted with a halogen, and the cycloalkylene, aralkylene and arylene may be substituted with a halogen, a C₁-C₁₀ alkyl or haloalkyl, or a C₁-C₁₀ alkoxyl or haloalkoxyl.

29. The process of embodiment 28, wherein X is F, Cl or Br; R¹ is H, a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R² is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R³ is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; and R⁴ is a C₁-C₆ straight or C₃-C₆ branched alkanediyl, a C₃-C₆ cycloalkylene, or a C₇-C₁₀ aralkylene, in which the alkanediyl may be substituted with a halogen, and the cycloalkylene and aralkylene may be substituted with a halogen, a C₁-C₁₀ alkyl or haloalkyl, or a C₁-C₁₀ alkoxyl or haloalkoxyl.

30. The process of embodiment 28, wherein X is F, Cl or Br; R¹ is H, a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R² is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R³ is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a Cl—C₆ alkyl or haloalkyl, or a C₁-C₆ alkoxyl or haloalkoxyl; and R⁴ is a C₁-C₄ straight or C₃-C₄ branched alkanediyl, a C₃-C₆ cycloalkylene, or a C₇-C₁₀ aralkylene, in which the alkanediyl may be substituted with a halogen, and the cycloalkylene and aralkylene may be substituted with a halogen, a C₁-C₆ alkyl or haloalkyl, or a C₁-C₆ alkoxyl or haloalkoxyl.

31. The process of embodiment 28, wherein X is F, Cl or Br; R¹ is H, a C₁-C₃ alkyl, or a C₃-C₆ cycloalkyl, in which the alkyl may be substituted with a halogen, and the cycloalkyl may be substituted with a halogen, a C₁-C₆ alkyl or haloalkyl; R² is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl; wherein R³ is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl; and R⁴ is a C₁-C₄ straight or C₃-C₄ branched alkanediyl, or a C₃-C₆ cycloalkylene, in which the alkanediyl may be substituted with a halogen, and the cycloalkylene may be substituted with a halogen, a C₁-C₆ alkyl or haloalkyl.

32. The process of embodiment 28, wherein X is F, Cl or Br; R¹ is H or a C₁-C₃ alkyl, in which the alkyl may be substituted with a halogen; R² is a C₁-C₄ straight or C₃-C₄ branched alkyl, in which the alkyl may be substituted with a halogen; R³ is a C₁-C₄ straight or C₃-C₄ branched alkyl, in which the alkyl may be substituted with a halogen; and R⁴ is a C₁-C₄ straight or C₃-C₄ branched alkanediyl, in which the alkanediyl may be substituted with a halogen.

33. The process of embodiment 28, wherein X is F, Cl or Br; R¹ is C₁-C₃ straight alkyl, in which the alkyl may be substituted with a halogen; R² is a C₃-C₄ branched alkyl, in which the alkyl may be substituted with a halogen; R³ is a C₃-C₄ branched alkyl, in which the alkyl may be substituted with a halogen; and R⁴ is a C₃-C₄ branched alkanediyl, in which the alkanediyl may be substituted with a halogen.

34. The process of embodiment 28, wherein X is Br; R¹ is methyl; R² is isopropyl; R³ is isopropyl, and R⁴ is 2,2-propandiyl.

35. The process of embodiment 28, wherein the halogenating agent in step i) is a chlorinating agent selected from the group consisting of NCS, Cl₂, dichlorodimethyl hydantoin, trichloroisocyanuric acid, N-chlorophthalimide, sulfuryl chloride and the mixtures thereof.

36. The process of embodiment 28, wherein the halogenating agent in step i) is a brominating agent selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid, bromodimethylsulfonium bromide, 5,5-dibromomeldrum's acid, bis(2,4,6-trimethylpyridine)-bromonium hexafluorophosphate, bromine monochloride and the mixtures thereof

37. The process of any one of embodiments 28-36, wherein the halogenating in step i) is carried out by oxyhalogenating process using an oxidizing agent and source of halogen ions under acidic conditions.

38. The process of embodiment 37, wherein the oxidizing agent is selected from the group consisting of hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, oxone, DMSO and mixtures thereof.

39. The process of any one of embodiments 37-38, wherein the source of halogen ions is a hydrogen halide or a mixture of a strong acid and an alkali metal or alkaline earth metal salt of a hydrogen halide.

40. The process of any one of embodiments 28-39, wherein a phase transfer catalyst is used in step ii).

41. The process of any one of embodiments 28-40, wherein a nitrite salt is used in step ii).

42. The process of embodiment 41, wherein the nitrite salt is selected from the group consisting of alkali metal nitrite salt and alkali earth metal nitrite salt.

43. The process of any one of embodiments 28-42, wherein steps i) and ii), steps ii) and iii), or steps i), ii), and iii) are carried out sequentially as a telescopic process.

44. The process of any one of embodiments 28-43, wherein the compound of Formula (I) is prepared according to the process of any one of embodiments 24-27.

45. Use of the compound of formula (V) as prepared according to any one of embodiments 8-22 for preparing isofetamid.

46. Use of the compound of formula (I) as prepared according to any one of embodiments 23-27 for preparing isofetamid.

47. Use of the compound of formula (X) as prepared according to any one of embodiments 28-44 for preparing isofetamid.

48. A process for preparation of isofetamid comprising: aa) preparing a compound of formula (I) according to the process of any one of embodiments 23-27; bb) preparing isofetamid from the compound of formula (I).

49. A process for preparation of isofetamid comprising: ai) preparing a compound of formula (V) according to the process of any one of embodiments 8-22; bi) preparing isofetamid from the compound of formula (V).

50. A process for preparation of isofetamid comprising: aj) preparing a compound of formula (X) according to the process of any one of embodiments 28-44; bj) preparing isofetamid from the compound of formula (X).

### DESCRIPTION OF SPECIFIC EMBODIMENTS OF THE INVENTION

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

“Halogen” refers in the present document to —F, —Cl, —Br or —I.

“Alkyl” means in the present document a straight or branched hydrocarbon chain radical consisting of carbon and hydrogen atoms, containing no unsaturation, having the number of carbon atoms indicated in each case, for example 1-16 carbon atoms (C₁-C₁₆—), which is attached to the rest of the molecule through a single bond. For example, an alkyl group comprises 1-8 carbon atoms, typically 1-4 carbon atoms. Exemplary alkyl groups can be methyl, ethyl, n-propyl, i-propyl, n-butyl, t-butyl, or n-pentyl.

“Haloalkyl” refers in the present document to an alkyl group that comprises one or more halogen substituents, that is, substituted with at least one of —F, —Cl, —Br or —I.

The skilled person is aware of different substituents used frequently in organic chemistry, such as haloalkyl groups comprising 1, 2, 3, 4, 5, 6, 7 or 8 halogen substituents. Haloalkyl groups wherein all positions have been substituted with halogen atoms are also known, for example, perfluoro or perchloro substituents. Exemplary haloalkyl groups can be —CH₂F, —CH₂Cl, —CHF₂, —CF₃, —CCl₃, or —CF₂CF₃.

“Cycloalkyl” means in the present document an alkyl group forming a closed ring and attached to the rest of the molecule through a single bond. Cycloalkyl groups can be substituted with other alkyl groups or form more than one ring. Exemplary cycloalkyl groups can be cyclopropyl, 2-mehtylcyclopropyl, cyclobutyl, cyclopentyl, cyclohexyl, 2-mehtylcyclohexyl, 4-mehtylcyclohexyl, cycloheptyl or cyclooctyl.

“Halocycloalkyl” refers in the present document to a cycloalkyl group that comprises one or more halogen substituents, that is, substituted with at least one of —F, —Cl, —Br or —I. The skilled person is aware of different substituents used frequently in organic chemistry, such as halocycloalkyl groups comprising 1, 2, 3, 4, 5, 6, 7 or 8 halogen substituents. Halocycloalkyl groups wherein all positions have been substituted with halogen atoms are also known, for example, perfluoro or perchloro substituents.

“Cyano” means in the present document —CN.

“Alkoxyl” means in the present document a radical of the formula —O-alkyl, wherein alkyl has been previously defined. Exemplary alkoxyl groups are methoxy, ethoxy or propoxy.

“Haloalkoxyl” refers in the present document to a radical of the formula —O— haloalkyl, for example —O—CH₂F, —O—CH₂Cl, —O—CHF₂, —O—CF₃, —O—CCl₃, —O—CF₂CF₃.

Aryl means in the present document radical (such as phenyl) derived from an aromatic hydrocarbon by the removal of one hydrogen atom from any ring atom.

Aralkyl means in the present document radical derived from an alkyl radical by replacing one or more hydrogen atoms by aryl.

Heteroaryl means in the present document radical derived from a heterocyclic aromatic hydrocarbon by the removal of one hydrogen atom from any ring atom.

“Alkanediyl” means in the present document a straight or branched hydrocarbon chain divalent radical consisting of carbon and hydrogen atoms, containing no unsaturation, having the number of carbon atoms indicated in each case, for example 1-16 carbon atoms (C₁-C₁₆—), which is attached to the rest of the molecule through a single bond. For example, an alkylene group comprises 1-8 carbon atoms, typically 1-4 carbon atoms. Exemplary alkylene groups can be methylene, ethylene, n-propylene, i-propylene, n-butylene, t-butylene, or n-pentylene.

“Cycloalkylene” means in the present document an alkanediyl group forming a closed ring and attached to the rest of the molecule through two single bonds. Cycloalkylene groups can be substituted with other alkyl groups or form more than one ring. Exemplary cycloalkylene groups can be cyclopropylene, 2-mehtylcyclopropylene, cyclobutylene, cyclopentylene, cyclohexylene, 2-mehtylcyclohexylene, 4-mehtylcyclohexylene, cycloheptylene, or cyclooctylene.

Arylene means in the present document a divalent radical (such as phenylene) derived from an aromatic hydrocarbon by the removal of two hydrogen atom from any aromatic ring.

Aralkylene means in the present document a divalent radical derived from an alkanediyl radical by replacing one or more hydrogen atoms by aryl.

It is understood that where a parameter range is provided, all integers within that range, and tenths thereof, are also provided by the invention as if the integers and tenths thereof are expressly described herein. For example, “0.1% to 70%” includes 0.1%, 0.2%, 0.3%, 0.4%, 0.5% etc. up to 70%.

The term “a” or “an” as used herein includes the singular and the plural, unless specifically stated otherwise. Therefore, the terms “a,” “an,” or “at least one” can be used interchangeably in this application.

Throughout the application, descriptions of various embodiments use the term “comprising”; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages, or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques. In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

### First Aspect

In the first aspect, the present disclosure provides a novel compound of Formula (V)

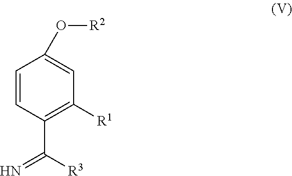

wherein:

R¹ is H, a C₁-C₁₀ (such as C₁-C₆, or even C₁-C₄) straight or C₃-C₁₀ (such as C₃-C₆) branched alkyl, a C₃-C₁₀ (such as C₃-C₆) cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl;

R² is a C₁-C₁₀ (such as C₁-C₆, or even C₁-C₄) straight or C₃-C₁₀ (such as C₃-C₆) branched alkyl, a C₃-C₁₀ (such as C₃-C₆) cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and

R³ is a C₁-C₁₀ (such as C₁-C₆, or even C₁-C₄) straight or C₃-C₁₀ (such as C₃-C₆) branched alkyl, a C₃-C₁₀ (such as C₃-C₆) cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl.

In one embodiment, R¹ is H, a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ (such as C₁-C₆, or even C₁-C₄) alkyl or haloalkyl, or a C₁-C₁₀ (such as C₁-C₆, or even C₁-C₄) alkoxyl or haloalkoxyl. In another embodiment, R¹ is H, a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl. In another embodiment, R¹ is H, a C₁-C₃ alkyl, or a C₃-C₆ cycloalkyl, in which the alkyl may be substituted with a halogen, and the cycloalkyl may be substituted with a halogen, a C₁-C₆ alkyl or haloalkyl. In another embodiment, R¹ is H or a C₁-C₃ alkyl, in which the alkyl may be substituted with a halogen. In another embodiment, R¹ is C₁-C₃ straight alkyl, in which the alkyl may be substituted with a halogen. In still another embodiment, R¹ is methyl.

In one embodiment, R² is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ (such as C₁-C₆, or even C₁-C₄) alkyl or haloalkyl, or a C₁-C₁₀ (such as C₁-C₆, or even C₁-C₄) alkoxyl or haloalkoxyl. In another embodiment, R² is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl. In another embodiment, R² is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl. In another embodiment, R² is a C₁-C₄ straight or C₃-C₄ branched alkyl, in which the alkyl may be substituted with a halogen. In another embodiment, R² is a C₃-C₄ branched alkyl, in which the alkyl may be substituted with a halogen. In still another embodiment, R² is isopropyl.

In one embodiment, R³ is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ (such as C₁-C₆, or even C₁-C₄) alkyl or haloalkyl, or a C₁-C₁₀ (such as C₁-C₆, or even C₁-C₄) alkoxyl or haloalkoxyl. In another embodiment, R³ is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl. In another embodiment, R³ is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl. In another embodiment, R³ is a C₁-C₄ straight or C₃-C₄ branched alkyl, in which the alkyl may be substituted with a halogen. In another embodiment, R³ is a C₃-C₄ branched alkyl, in which the alkyl may be substituted with a halogen. In still another embodiment, R³ is isopropyl.

We have now found that the compound of Formula (V) may be used as alternative intermediates in the synthetic pathway to prepare the compound of Formula (I) and isofetamid.

### Second Aspect

In the second aspect, the present disclosure also provides a process for preparing a compound of Formula (V) comprising:
b) reacting a compound of Formula (III) with a compound of Formula (IV)

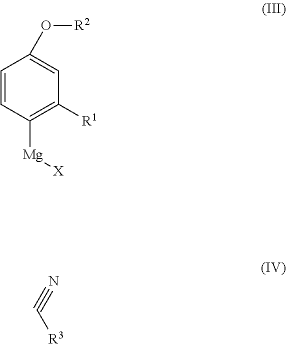

or
b1) reacting a compound of Formula (XII) with a compound of Formula (XIII)

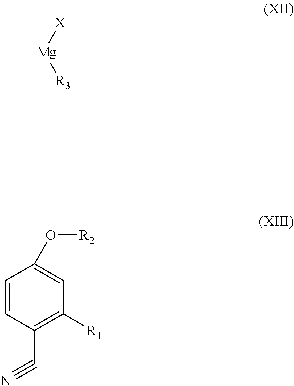

to prepare a compound of Formula (V);

wherein:

X is a halogen, and

R¹, R², and R³ are defined as in the first aspect.

In one embodiment, X is F, Cl, Br or I. In another embodiment X is F, Cl or Br. In another embodiment X is Cl or Br. In still another embodiment, X is Br.

In the second aspect, R¹, R², and R³ are defined as in the first aspect. Therefore, unless otherwise indicated, all the specific descriptions on R¹, R², and R³ in the first aspect apply to here in the second aspect as all relevant specific descriptions have been copied here.

In some embodiments, when the process of the second aspect comprises a step b), the process may further comprise a step a) reacting the compound of Formula (II) with magnesium to prepare the compound of Formula (III)

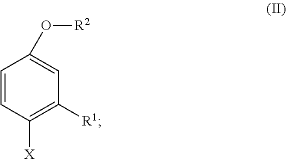

wherein: X, R¹, and R² are defined as mentioned above for the compound of Formula (III).

In some embodiments, when the process of the second aspect comprises a step b1), the process may further comprise a step al) reacting the compound of Formula (XI) with magnesium to prepare the compound of Formula (XII)

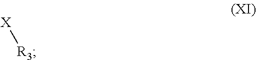

wherein: X and R³ are defined as mentioned above for the compound of Formula (XII).

In step a), a compound of Formula (II) is reacted with magnesium, optionally in the presence of organic solvent to form a compound of Formula (III), optionally in the presence of an inert gas (e.g., N₂), and optionally in the presence of an initiator. In step b), the resulting compound of Formula (III) is reacted with a cyano compound of Formula (IV) to form an imine compound of Formula (V).

In step a), the optional organic solvent may be solvents usual for this reaction, like etheric solvents, for example alkyl or cycloalkyl ethers, e.g., diethyl ether, methyl tert-butyl ether, methyl cyclopentyl ether, THF, 2-methyl THF etc., and/or their mixtures with aliphatic or aromatic solvents like toluene or petrol ether. Preferably, the solvent is tetrahydrofuran. If present, the mol ratio of the etheric solvents to the compound of Formula (II) should be no less than 1:1, and preferably 2:1 and more. If present, the weight ratio of the organic solvent (including mixed solvents) to the compound of Formula (II) may be from 0.1:1 to 10:1, and preferably from 1:1 to 5:1, more preferably from 3:1 to 5:1. The reaction temperature may be from 0° C. up to 150° C. or the boiling point of the solvent, preferably from 20° C. up to 70° C. (preferably 50 to 60° C.) or boiling point of the solvent. The reaction time in this step is typically from 2 to 20 hours, preferably from 4 to 8 hours. The mol ratio of Mg to the compound of Formula (II) is from 1:1 to 10:1, and preferably is from 1:1 to 2:1.

In step a), the optional initiator may be initiator usual for this reaction, like Iodine, alkyl magnesium bromide, dibromoethane, etc. Preferably, the initiator is methyl magnesium bromide. If present, the mol percent of the initiators to the compound of Formula (II) should be from 0.5 to 5 mol %, but preferably from 1 to 3 mol % based on the compound of Formula (II).

In step b), optionally, organic solvents can be used, which have to be aprotic and not reacting with Grignard reagent. Preferably, the solvent used in step (b) may be etheric solvents, for example alkyl or cycloalkyl ethers, e.g., diethyl ether, methyl tert-butyl ether, methyl cyclopentyl ether, THF, 2-methyl THF etc., and/or their mixtures with aliphatic or aromatic solvents. Preferably, steps a) and b) are carried out as a one pot process. Preferably, the solvent is tetrahydrofuran. If present, the mol ratio of the etheric solvents to the compound of Formula (III) should be at an amount of not less than 1:1, and preferably 2:1 and more. If present, the weight ratio of the organic solvent (including mixed solvents) to the compound of Formula (III) may be from 0.1:1 to 10:1, preferably from 1:1 to 5:1, and more preferably from 3:1 to 5:1. The reaction temperature is typically between 0° C. and 150° C. or the boiling point of the solvent, preferably from 15° C. up to 60° C. or boiling point of the solvent. The reaction time is typically from 2 to 20 hours, preferably from 4 to 8 hours. The molar ratio of compound of Formula (III) to a compound of Formula (IV) is from 1:1 to 1:3, and preferably is from 1:1 to 1:1.3. If necessary, the desired product, a compound of Formula (V), can be isolated by methods known to those skilled in the art, including crystallization, extraction and distillation.

In step a1), a compound of Formula (XI) is reacted with magnesium, optionally in the presence of organic solvent to form a compound of Formula (XII), optionally in the presence of an inert gas (e.g., N₂), and optionally in the presence of an initiator. In step a1), the optional organic solvent may be solvents usual for this reaction, like etheric solvents, for example alkyl or cycloalkyl ethers, e.g., diethyl ether, methyl tert-butyl ether, methyl cyclopentyl ether, THF, 2-methyl THF etc., and/or their mixtures with aliphatic or aromatic solvents like toluene or petrol ether. Preferably, the solvent is tetrahydrofuran. If present, the mol ratio of the etheric solvents to the compound of Formula (XI) should be no less than 1:1, and preferably 2:1 and more. If present, the weight ratio of the organic solvent (including mixed solvents) to the compound of Formula (XI) may be from 0.1:1 to 10:1, and preferably from 1:1 to 5:1, more preferably from 3:1 to 5:1. The reaction temperature may be from 0° C. up to 150° C. or the boiling point of the solvent, preferably from 20° C. up to 70° C. (preferably 50 to 60° C.) or boiling point of the solvent. The reaction time in this step is typically from 2 to 20 hours, preferably from 4 to 8 hours. The mol ratio of Mg to the compound of Formula (XI) is from 1:1 to 10:1, and preferably is from 1:1 to 2:1. In step a1), the optional initiator may be initiator usual for this reaction, like Iodine, alkyl magnesium bromide, dibromoethane, etc. Preferably, the initiator is methyl magnesium bromide. If present, the mol percent of the initiators to the compound of Formula (XI) should be from 0.5 to 5 mol %, but preferably from 1 to 3 mol % based on the compound of Formula (XI).

In step b1), alternatively, the compound of Formula (V) may be prepared by reacting a compound of Formula (XII) with a compound of Formula (XIII). Optionally, organic solvents can be used in step b1), which have to be aprotic and not reacting with Grignard reagent. Preferably, the solvent used in step (b1) may be etheric solvents, for example alkyl or cycloalkyl ethers, e.g., diethyl ether, methyl tert-butyl ether, methyl cyclopentyl ether, THF, 2-methyl THF etc., and/or their mixtures with aliphatic or aromatic solvents like toluene or petrol ether. Preferably, the solvent is tetrahydrofuran. If present, the mol ratio of the etheric solvents to the compound of Formula (XI) should be at an amount of not less than 1:1, and preferably 2:1 and more. If present, the weight ratio of the organic solvent (including mixed solvents) to the compound of Formula (XII) may be from 0.1:1 to 10:1, preferably from 1:1 to 5:1, and more preferably from 3:1 to 5:1. The reaction temperature is typically between 0° C. and 150° C. or the boiling point of the solvent, preferably from 15° C. up to 80° C. or boiling point of the solvent. The reaction time is typically from 2 to 20 hours, preferably from 4 to 8 hours. The molar ratio of compound of Formula (XII) to a compound of Formula (XIII) is from 1:1 to 1:3, and preferably is from 1:1 to 1:1.3. If necessary, the desired product, a compound of Formula (V), can be isolated by methods known to those skilled in the art, including crystallization, extraction and distillation.

The solvents used in steps a) and b) may be the same or different. The solvents used in step a1) and b1) may be the same or different.

In some embodiments, the steps a) and b) may be done sequentially as a telescopic process without intermediate separation. That is, the compound of Formula (III) may not be isolated. Instead, the reaction mixture obtained in step a) may be used directly as it is in the next step b).

In some embodiments, the steps a1) and b1) may be done sequentially as a telescopic process without intermediate separation. That is, the compound of Formula (XII) may not be isolated. Instead, the reaction mixture obtained in step a1) may be used directly as it is in the next step b1).

In some embodiments, the process of second aspect may further comprises:
d) reacting the compound of Formula (VI) with an alkylating agent

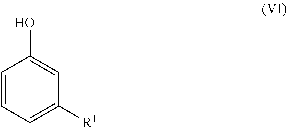

to prepare a compound of Formula (VII)

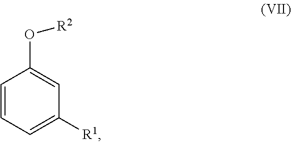

e) halogenating the compound of Formula (VII) with a halogenating agent to prepare the compound of Formula (II),

wherein, R¹ and R² are defined above for the compound of Formula (II).

In step d), a compound of Formula (VI) is reacted with an alkylating agent like alkyl halide (e.g., alkyl chloride, like n-butyl or sec-butyl chlorides), and alkyl bromide (like 2-bromopropane or methyl bromide), dialkyl sulfate (like dimethyl or diethyl sulfates), alkene (like propylene) etc., optionally in the presence of an organic solvent to form the compound of Formula (VII). The molar ratio of compound of Formula (VI) to the alkylating agent is from 1:1 to 1:2, and preferably is from 1:1 to 1:1.3.

In case step d) is carried out with alkyl halide (e.g., bromide) and in the presence of a base, at the end of the process reaction mixture contains a halide (e.g., bromide) salt. In this case step e) may be done by oxyhalogenation (e.g., oxybromination) process by adding suitable acid and oxidant, for example sulfuric acid and hydrogen peroxide. As such step e) may be a direct continuation of step d) and may be performed in the same vessel.

In step d), as the optional organic solvent, both polar and non-polar organic solvents can be used, wherein among polar solvents C₁-C₆ alcohols (e.g., methanol, ethanol), acetonitrile, tetrahydrofuran, N,N-dimethylformamide, dimethyl sulfoxide, and the like are suitable. Among non-polar solvents toluene, chlorobenzene, dichloromethane, dichloroethane, chloroform and the like are suitable. Two or more of the above-mentioned solvents may be used as a mixture, and the reaction may be performed in a single-phase system or a two-phase system. If present, the weight ratio of the organic solvents to the compound of Formula (VI) is from 1:1 to 10:1, preferably from 2:1 to 5:1. Alkylating agents may be used as a solvent too.

In some embodiments, step d) includes the use of a base to prepare the compound of Formula (VII). The base according to the above process is selected from the group comprising of alkali metal hydroxides, e.g. LiOH, NaOH or KOH, alkali metal carbonates, e.g. Li₂CO₃, Na₂CO₃, K₂CO₃ or Cs₂CO₃, hydrides (e.g., NaH), alkaline earth metal hydroxides, e.g. Mg(OH)₂ or Ca(OH)₂ and alkaline earth metal carbonates, e.g. MgCO₃ or CaCO₃, sodium methoxide, potassium methoxide, sodium ethoxide, potassium ethoxide, potassium tert-butoxide, lithium tert-butoxide, sodium bicarbonate, potassium bicarbonate, and the mixtures thereof. If present, the mol ratio of the base to the compound of Formula (VI) may be from 1:1 to 5:1, preferably from 1.1:1 to 1.5:1.

In step d), the reaction temperature is typically between 0° C. and 150° C. or the boiling point of the solvent, preferably from 20 to 100° C. or boiling point of the solvent. The reaction time is typically from 1 to 20 hours, preferably from 2 to hours. The desired product, a compound of Formula (VII), can be isolated by methods known to those skilled in the art, including crystallization, extraction and distillation.

In step e), the resulting compound of Formula (VII) undergoes halogenation (e.g., bromination) to form the compound of Formula (II). In some embodiments, the halogenating agent used in step e) may be a chlorinating agent, a brominating agent or an iodinating agent. In some embodiments, the halogenating agent is a brominating agent, and the brominating agent according to the above process is selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid, bromodimethylsulfonium bromide, 5,5-dibromomeldrum's acid, bis(2,4,6-trimethylpyridine)-bromonium hexafluorophosphate, bromine monochloride and the mixtures thereof. In some embodiments, the halogenating agent is a chlorinating agent, and the chlorinating agent according to the above process is selected from the group consisting of NCS, Cl₂, dichlorodimethyl hydantoin, trichloroisocyanuric acid, N-chlorophthalimide, sulfuryl chloride and the mixtures thereof. The molar ratio of the halogenating agent to the compound of Formula (VI) may be from 0.9:1 to 1.5:1, preferably from 1:1 to 1.2:1.

In some embodiments, the halogenating in step e) may be carried out by an oxyhalogenating process using suitable oxidizing agent and source of halogen ions under acidic conditions. A halogenating agent will be produced with suitable oxidizing agent and source of halogen ions under acidic conditions. As a source of halogen ions a hydrogen halide like HBr or HCl or an alkali metal or alkaline earth metal salt thereof in a mixture with a strong acid may be used. Here, alkali metal or alkaline earth metal may be Li, K, Na, Cs, Mg or Ca. The strong acid is for example H₂SO₄, CF₃SO₃H, H₃PO₄, HNO₃. The mol ratio of the hydrogen halide or the alkali metal or alkaline earth salt thereof to the compound of Formula (VII) may be from 0.9:1 to 1.5:1, preferably from 1:1 to 1.2:1. The mol ratio of the strong acid if present to the compound of Formula (VII) may be from 0.9:1 to 1.5:1, preferably from 1:1 to 1.2:1.

The oxidizing agent may be selected from the group consisting of hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, oxone, DMSO, and mixtures thereof. The mol ratio of the oxidizing agent to the compound of Formula (VI) may be from 0.9:1 to 1.5:1, preferably from 1:1 to 1.2:1.

The acidic conditions may also be produced by the presence of as the source of halogen ions a hydrogen halide like HBr or HCl as mentioned above or the presence of a strong acid like H₂SO₄ in case an alkali metal or alkaline earth metal salt of a hydrogen halide like KCl and NaBr is used as a source of halogen ions.

A phase transfer catalyst may also be used in the steps d) and/or e) depending on solvents and reagent used in the steps. The phase transfer catalyst may be selected from the group comprising ammonium salts (e.g. benzyltrialkylammonium halides such as benzyldimethyldecylammonium chloride, or tetraalkylammonium halides such as methyltrioctylammonium chloride, tetrabutyl ammonium bromide (TBAB) or tetrabutyl ammonium iodide (TBAI)), heterocyclic ammonium salts (e.g. 1-butyl-2,3-dimethylimidazolium tetrafluoroborate or Hexadecylpyridinium bromide), nonionic phase transfer catalysts (e.g. crown-ethers, polyethylene glycols, modified tocopherols such as DL-α-tocopherol methoxypolyethyleneglycol succinate) and phosphonium salts (e.g. tetraphenylphosphonium chloride or trihexyltetradecylphosphonium bromide). In the step d), the phase transfer catalyst may be used at an amount of 1 to 5 mol % based on the compound of Formula (VI). In the step e), the phase transfer catalyst may be used at an amount of 1 to 5 mol % based on the compound of Formula (VII).

In step e), organic solvent may also be used which may be polar or non-polar. Among polar solvents C₁-C₆ alcohols (e.g., methanol, ethanol), acetonitrile, N,N-dimethylformamide, and the like are suitable. Among non-polar solvents chlorobenzene, dichloromethane, dichloroethane, chloroform and the like are suitable. Two or more of the above-mentioned solvents may be used as a mixture, and the reaction may be performed in a single-phase system or a two-phase system. If present, the organic solvents are present at an amount of from 1:1 to 10:1 weight ratio, preferably from 2:1 to 5:1 based on the compound of Formula (VII).

In step e), the reaction temperature is typically between 0° C. and 80° C. or the boiling point of the solvent, preferably from 0 to 30° C. The reaction time is typically from 1 to 20 hours, preferably from 3 to 12 hours. The desired product, a compound of Formula (II), can be isolated by methods known to those skilled in the art, including crystallization, extraction and distillation.

In some embodiments, steps d) and e) may be done sequentially in one reaction vessel, namely as a one-pot process. In some embodiments, the steps d) and e) may be done sequentially in telescopic manner. That is, the compound of Formula (VII) may not be isolated. Instead, the reaction mixture obtained in step d) may be used directly as it is in the next step e).

In some embodiments, the steps d), e), a), b) and c) may be done sequentially in telescopic manner without intermediates separation.

In the present disclosure, unless otherwise indicated, all the specific descriptions made in the first aspect apply to the second aspect as all relevant descriptions have been copied here. For example, unless otherwise indicated, all the specific descriptions on R¹, R², and R³ in the first aspect apply to the second aspect as all relevant specific descriptions have been copied in the corresponding places in the second aspect.

### Third Aspect

In the third aspect, the present disclosure also provides a process for preparing a compound of Formula (I),

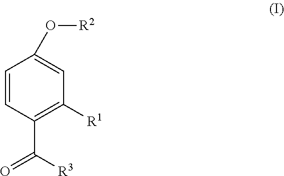

comprising:
B) preparing the compound of Formula (V) according to the process specified in the second aspect;

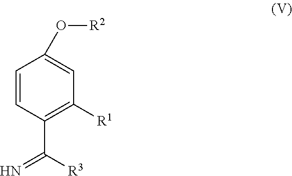

c) hydrolyzing the resulting compound of Formula (V);

wherein: X, R¹, R², and R³ are defined as in the first aspect or second aspect for Formula (V).

In the third aspect, step B) represents preparing the compound of Formula (V) according to the process specified in the second aspect. Therefore, all the specific descriptions made in the second aspects apply to step B) as all relevant descriptions have been copied here. For example, unless otherwise indicated, all the specific descriptions on steps a), b), d), e), a1), b1), and relevant materials used therein (e.g., bases, solvents, etc.), conditions (e.g., temperature and time, etc.) and the like specified in the second aspect apply to here in the third aspect as all relevant specific descriptions have been copied here.

In step c), the compound of Formula (V) obtained in step B) undergoes hydrolysis. The hydrolysis may be either acid-catalyzed hydrolysis or base-catalyzed hydrolysis. Suitable acids and bases are known in the art. For example, suitable acids for hydrolysis may be, but are not limited to, from “super” acids like Triflic acid, including inorganic acids like hydrochloric acid, sulfuric acid, phosphoric acid up to carboxylic acids like trifluoroacetic acid, formic acid, and benzoic acid. The mol ratio of the acid to the compound of Formula (V) may be from 1:1 to 10:1, preferably from 1.5:1 to 2.5:1. For example, suitable bases for hydrolysis may include, but are not limited to, both organic bases like tertiaries amines like triethyl amine or pyridine and inorganic bases like sodium or potassium hydroxide or carbonates, Mg, Ca, Ba hydroxides or carbonates. The mol ratio of the base to the compound of Formula (V) may be from 0.1:1 to 10:1.

In step c), optionally, organic solvents can be used which have to be stable under hydrolysis conditions. Preferably organic solvents may be etheric solvents, like alkyl or cycloalkyl ethers, e.g., diethyl ether, methyl tert-butyl ether, methyl cyclopentyl ether, THF, 2-methyl THF etc., and/or their mixtures with aliphatic or aromatic solvents like toluene or petrol ether. Preferably steps a), b) and c) are carried out as a telescopic process without intermediate separation. C₁-C₆ alcohols (e.g., methanol, ethanol) and non-polar solvents may also be used in step c). Among non-polar solvents toluene, chlorobenzene, dichloromethane, dichloroethane, chloroform and the like are suitable. DMSO may be used for basic hydrolysis only. The reaction may be performed in a single-phase system or a two-phase system. Preferably, the solvent is tetrahydrofuran. If present, the weight ratio of the organic solvent (including mixed solvents) to the compound of Formula (V) may be from 1:1 to 10:1, and preferably from 3:1 to 5:1.

In step (c), water is present at not less than 1 mol per mol of the compound of Formula (V) up to 10 weights per weight of the compound of Formula (V).

The solvents used in step a) and b), steps b) and c), step a) and c), and steps a), b) and c) may be the same or different. The solvents used in step a1) and b1), steps b1) and c), step a1) and c), and steps a1), b1) and c) may be the same or different.

The reaction temperature in step c) is typically between 0° C. and 100° C. or the boiling point of the solvent, preferably from 10 to 80° C. or boiling point of the solvent. The reaction time is typically from 2 to 20 hours, preferably from 3 to 8 hours. If necessary, the desired product, a compound of Formula (I), can be isolated by methods known to those skilled in the art, including crystallization, extraction and distillation.

In some embodiments, the steps a) and b), steps b) and c), or steps a), b) and c) may be done sequentially as a telescopic process without intermediate separation. That is, the compound of Formula (III) and/or the compound of Formula (V) may not be isolated. Instead, the reaction mixture obtained in step a) and/or b) may be used directly as it is in the next step b) and/or c).

In some embodiments, the steps a1) and b1), steps b1) and c), or steps a1), b1) and c) may be done sequentially as a telescopic process without intermediate separation. That is, the compound of Formula (XII) and/or the compound of Formula (V) may not be isolated. Instead, the reaction mixture obtained in step a1) and/or b1) may be used directly as it is in the next step b1) and/or c).

We have now found novel compounds, such as the compound of Formula (V) which may be used as alternative intermediates in the synthetic pathway to prepare the compound of Formula (I) and isofetamid. The novel synthetic pathway allows the synthesis of the compound of Formula (I) or analogs thereof without the need for isolation between the individual steps.

The compound of Formula (I) is an important intermediate and is used in the preparation of isofetamid, as described in WO 2006/016708 incorporated herein by reference in its entirety.

In the present disclosure, unless otherwise indicated, all the specific descriptions made in the first and second aspects apply to the third aspect as all relevant descriptions have been copied here. For example, unless otherwise indicated, all the specific descriptions on X, R¹, R², and R³ specified in the first aspect and second aspect apply to the third aspect as all relevant specific descriptions have been copied here; and all the specific descriptions on steps a), b), d), e), a1), b1), and relevant materials used therein (e.g., bases, solvents, etc.), conditions (e.g., temperature and time, etc.) and the like specified in the second aspect apply to the third aspect as all relevant specific descriptions have been copied in the corresponding places in the third aspect.

### Fourth Aspect

In the fourth aspect, the present disclosure also provides a process for preparing a compound of Formula (X) comprising:
i. halogenating a compound of Formula (I) with a halogenating agent to prepare a compound of Formula (VIII)

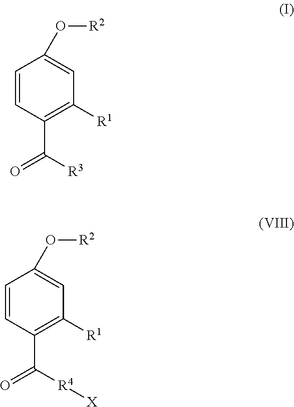

ii. substitution in the compound of Formula (VIII) to prepare a compound of Formula (IX)

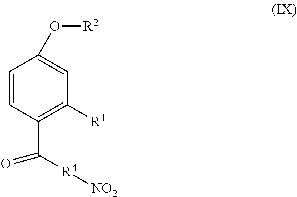

iii. reducing the compound of Formula (IX) to prepare a compound of Formula (X)

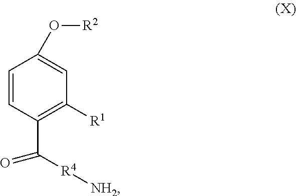

wherein: X, R¹, R² and R³ are defined as in the first aspect and the second aspect, and R⁴ is a C₁-C₁₀ (such as C₁-C₆, or even C₁-C₄) straight or C₃-C₁₀ (such as C₃-C₆, or even C₃-C₄) branched alkanediyl, a C₃-C₁₀ cycloalkylene, a C₇-C₁₀ aralkylene, or a C₆-C₁₀ arylene, in which the alkanediyl may be substituted with a halogen, and the cycloalkylene, aralkylene and arylene may be substituted with a halogen, a C₁-C₁₀ (such as C₁-C₆, or even C₁-C₄) alkyl or haloalkyl, or a C₁-C₁₀ (such as C₁-C₆, or even C₁-C₄) alkoxyl or haloalkoxyl.

In the fourth aspect, X, R¹, R² and R³ are defined as in the first aspect and the second aspect. Unless otherwise indicated, all the specific descriptions on X, R¹, R², and R³ in the first and second aspects apply to here in the fourth aspect as all relevant specific descriptions have been copied here.

In the fourth aspect, in one embodiment, R⁴ is a C₁-C₆ straight or C₃-C₆ branched alkanediyl, a C₃-C₆ cycloalkylene, or a C₇-C₁₀ aralkylene, in which the alkanediyl may be substituted with a halogen, and the cycloalkylene and aralkylene may be substituted with a halogen, a C₁-C₁₀ (such as C₁-C₆, or even C₁-C₄) alkyl or haloalkyl, or a C₁-C₁₀ (such as C₁-C₆, or even C₁-C₄) alkoxyl or haloalkoxyl. In another embodiment, R⁴ is a C₁-C₄ straight or C₃-C₄ branched alkanediyl, a C₃-C₆ cycloalkylene, or a C₇-C₁₀ aralkylene, in which the alkanediyl may be substituted with a halogen, and the cycloalkylene and aralkylene may be substituted with a halogen, a C₁-C₆ alkyl or haloalkyl, or a C₁-C₆ alkoxyl or haloalkoxyl. In another embodiment, R⁴ is a C₁-C₄ straight or C₃-C₄ branched alkanediyl, or a C₃-C₆ cycloalkylene, in which the alkanediyl may be substituted with a halogen, and the cycloalkylene may be substituted with a halogen, a C₁-C₆ alkyl or haloalkyl. In another embodiment, R⁴ is a C₁-C₄ straight or C₃-C₄ branched alkanediyl, in which the alkanediyl may be substituted with a halogen. In another embodiment, R⁴ is a C₃-C₄ branched alkanediyl, in which the alkanediyl may be substituted with a halogen. In still another embodiment, R⁴ is 2,2-propandiyl.

In step i), a compound of Formula (I) is halogenated (for example brominated) with a halogenating agent, optionally in the presence of organic solvent to form a compound of Formula (VIII). As the solvents, polar and non-polar organic solvents can be used. Among polar solvents C₁-C₆ alcohols (e.g., methanol, ethanol), acetonitrile, tetrahydrofuran, acetic acid, dimethyl sulfoxide and the like are suitable. Among non-polar solvents toluene, chlorobenzene, dichloromethane, ethyl acetate, dichloroethane, chloroform and the like are suitable. Two or more of the above-mentioned solvents may be used as a mixture, and the reaction may be performed in a single-phase system or a two-phase system. Preferably, the solvent is DMSO, acetic acid or ethyl acetate. If present, the weight ratio of the organic solvents to the compound of Formula (I) is from 10:1 to 1:1, preferably from 3:1 to 1:1. The reaction temperature is typically between 0° C. and 150° C. or the boiling point of the solvent, and preferably from 50 to 90° C. or the boiling point of the solvent. The reaction time is typically from 2 to 20 hours, and preferably from 2 to 8 hours. The desired product, a compound of Formula (VIII), can be isolated by methods known to those skilled in the art, including crystallization, extraction and distillation.

In some embodiments, the halogenating agent used in step i) may be a chlorinating agent, a brominating agent or an iodinating agent. In some embodiments, the halogenating agent is a brominating agent, and the brominating agent according to the above process is selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid, bromodimethylsulfonium bromide, 5,5-dibromomeldrum's acid, bis(2,4,6-trimethylpyridine)-bromonium hexafluorophosphate, bromine monochloride and the mixtures thereof. In some embodiments, the halogenating agent is a chlorinating agent, and the chlorinating agent according to the above process is selected from the group consisting of NCS, Cl₂, dichlorodimethyl hydantoin, trichloroisocyanuric acid, N-chlorophthalimide, sulfuryl chloride and the mixtures thereof. The molar ratio of the halogenating agent to the compound of Formula (I) may be from 0.9:1 to 1.5:1, preferably from 1:1 to 1.2:1.

In some embodiments, the halogenating in step i) may be carried out by an oxyhalogenating process using suitable oxidizing agent and source of halogen ions under acidic conditions. A halogenating agent will be produced with suitable oxidizing agent and source of halogen ions under acidic conditions. As a source of halogen ions a hydrogen halide like HBr or HCl or an alkali metal or alkaline earth metal salt thereof in a mixture with a strong acid may be used. Here, alkali metal or alkaline earth metal may be Li, K, Na, Cs, Mg or Ca. The mol ratio of the hydrogen halide or the alkali metal or alkaline earth salt thereof to the compound of Formula (I) may be from 0.9:1 to 1.5:1, preferably from 1:1 to 1.2:1. The mol ratio of the strong acid if present to the compound of Formula (I) may be from 0.9:1 to 1.5:1, preferably from 1:1 to 1.2:1.

The oxidizing agent may be selected from the group consisting of hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, oxone, DMSO, and mixtures thereof. The mol ratio of the oxidizing agent to the compound of Formula (I) may be from 0.9:1 to 1.5:1, preferably from 1:1 to 1.2:1.

The acidic conditions is also produced by the presence of as the source of halogen ions a hydrogen halide like HBr or HCl as mentioned above or the presence of a strong acid like H₂SO₄ in case alkali metal or alkaline earth metal salt of a hydrogen halide like KCl and NaBr is used as a source of halogen ions.

In step ii), the resulting compound of Formula (VIII) undergoes a substitution reaction to form the compound of Formula (IX).

In step ii), optionally, organic solvents can be used which may be polar or non-polar. Among polar solvents C₁-C₆ alcohols (e.g., methanol, ethanol), acetonitrile, tetrahydrofuran, N,N-dimethylformamide, dimethyl sulfoxide and the like are suitable. Among non-polar solvents toluene, chlorobenzene, ethyl acetate, dichloromethane, dichloroethane, chloroform and the like are suitable. Two or more of the above-mentioned solvents may be used as a mixture, and the reaction may be performed in a single-phase system or a two-phase system. Preferably, the solvent is DMSO. If present, the weight ratio of the organic solvents to the compound of Formula (VIII) in step ii) is 10:1 to 1:1, preferably 3:1 to 1:1.

In some embodiments, for the substitution reaction, step ii) includes the use of a nitrite salt to prepare the compound of Formula (IX). The nitrite salt may be selected from the group consisting of alkali metal (for example, Na, K, Li) nitrite salt and alkali earth metal (for example, Mg, Ca, Ba) nitrite salt. The molar ratio of the nitrite salt to the compound of Formula (VIII) is 1:1 to 3:1.

In some embodiments, step ii) may include the use of a phase transfer catalyst. The phase transfer catalyst may be selected from the group comprising ammonium salts (e.g. benzyltrialkylammonium halides such as benzyldimethyldecylammonium chloride, or tetraalkylammonium halides such as methyltrioctylammonium chloride, tetrabutyl ammonium bromide (TBAB) or tetrabutyl ammonium iodide (TBAI)), heterocyclic ammonium salts (e.g. 1-butyl-2,3-dimethylimidazolium tetrafluoroborate or Hexadecylpyridinium bromide), nonionic phase transfer catalysts (e.g. crown-ethers, polyethylene glycols, modified tocopherols such as DL-α-tocopherol methoxypolyethylene glycol succinate) and phosphonium salts (e.g. tetraphenylphosphonium chloride or trihexyltetradecylphosphonium bromide). In step ii), if present, the phase transfer catalyst is used at an amount of 0.5 to 5 mol % based on the compound of Formula (VIII).

In step ii), the reaction temperature is typically between 0° C. and 100° C. or the boiling point of the solvent, preferably from 10 to 50° C. or boiling point of the solvent. The reaction time is typically from 2 to 20 hours, preferably from 2 to 8 hours.

If necessary, the desired product, a compound of Formula (IX) can be isolated by methods known to those skilled in the art, including crystallization, extraction and distillation.

In some embodiments, the steps i) and ii) may be done sequentially by telescopic manner without intermediate separation.

In step iii), the resulting compound of Formula (IX) is reduced to form the compound of Formula (X). To reduce the nitro group on the compound of Formula (IX) to an amine group, conventional method may be used. In one embodiment, the compound of Formula (IX) may be reacted with a reducing agent like lithium aluminum hydride, lithium borohydride or sodium borohydride, sodium hydrosulfite or sodium sulfide, trimethylsilyl chloride, Fe, Zn, Tin(II) chloride, hydrogen in the presence of metal catalysts based on Pt, Pd, Rh, Ni and so on. Thus, the reaction of the compounds of Formula (IX) with reducing agents provides the compounds of Formula (X), which are key intermediates in the synthesis of active ingredients used in agriculture. In some embodiments, the reducing agent Fe may be in the form of iron powder, iron shavings, iron mud, and a mixture thereof. In some embodiments, the reducing agent Fe may be in the form of a mixture of iron powder and iron mud. The equivalent ratio of the reducing agent to the compound of Formula (IX) may be from 2:1 to 20:1.

In step iii), optionally, organic solvents can be used which may be polar or non-polar. Among polar solvents C₁-C₆ alcohols (e.g., methanol, ethanol), acetonitrile, tetrahydrofuran, N,N-dimethylformamide, acetic acid, ethyl acetate and the like are suitable. Among non-polar solvents toluene, chlorobenzene, dichloromethane, dichloroethane, chloroform, MIBK and the like are suitable. Two or more of the above-mentioned solvents may be used as a mixture, and the reaction may be performed in a single-phase system or a multi-phase system. Preferably, the solvent is ethyl acetate. If present, the weight ratio of the organic solvents to the compound of Formula (IX) in step iii) is from 1:1 to 10:1.

In step iii), the reaction temperature is typically between −20° C. and 100° C. or the boiling point of the solvent, preferably from 0 to 60° C. or boiling point of the solvent. The reaction time is typically from 2 to 30 hours, preferably from 3 to hours. If necessary, the desired product, a compound of Formula (X), can be isolated by methods known to those skilled in the art, including crystallization, extraction and distillation.

In some embodiments, the steps ii) and iii) may be done sequentially in telescopic manner without intermediates separation.

In some embodiments, the steps i), ii), and iii) may be done sequentially in telescopic manner without intermediates separation.

In some embodiments, the compound of Formula (I) is prepared or has been prepared according to the process as described in the third aspect. Therefore, unless otherwise indicated, all the specific descriptions made in the third aspects regarding the process for preparing the compound of Formula (I), step B), and step c) apply to here in the fourth aspect as all relevant descriptions have been copied here. Similarly, regarding step B), unless otherwise indicated, all the specific descriptions made in the second aspect also apply here in the fourth aspect as all relevant descriptions have been copied here. For example, unless otherwise indicated, all the specific descriptions on steps B), c), and relevant materials used therein (e.g., bases, solvents, etc.), conditions (e.g., temperature and time, etc.) and the like specified in third aspect apply to the fourth aspect as all relevant specific descriptions have been copied here. For another example, all the specific descriptions on steps a), b), d), e), a1), b1), and relevant materials used therein (e.g., bases, solvents, etc.), conditions (e.g., temperature and time, etc.) and the like specified in the second aspect apply to the fourth aspect as all relevant specific descriptions have been copied here.

The provided processes represent environmentally-friendly alternatives to previously disclosed methods of preparation, reducing solvent waste and generating innocuous byproducts.

In the present disclosure, unless otherwise indicated, all the specific descriptions made in the first, second and third aspects apply to the fourth aspect as all relevant descriptions have been copied here. For example, unless otherwise indicated, all the specific descriptions on X, R¹, R², and R³ specified in the first aspect and the second aspect apply to the fourth aspect as all relevant specific descriptions have been copied here; all the specific descriptions on steps a), b), c), d), e), a1), b1), and relevant materials used therein (e.g., bases, solvents, etc.), conditions (e.g., temperature and time, etc.) and the like specified in the second aspect apply to the fourth aspect as all relevant specific descriptions have been copied here; and all the specific descriptions on steps B), c), and relevant materials used therein (e.g., bases, solvents, etc.), conditions (e.g., temperature and time, etc.) and the like specified in the third aspect apply to the fourth aspect as all relevant specific descriptions have been copied in the corresponding places in the fourth aspect.

### The Fifth and Other Aspects

In the fifth aspect, the present disclosure provides use of the compound of formula (V) as prepared according to the process as described in the second aspect for preparing isofetamid.

In a further aspect, the present disclosure provides use of the compound of formula (I) as prepared according to the process as described in the third aspect for preparing isofetamid.

In a further aspect, the present disclosure provides use of the compound of formula (X) as prepared according to the process as described in the fourth aspect for preparing isofetamid.

In a further aspect, the present disclosure provides a process for preparation of isofetamid comprising:
aa) preparing compound of formula (I) according to the process as described in the third aspect;
bb) providing reaction conditions for preparation of isofetamid.

According to an embodiment the reaction conditions in step bb) include but are not limited to nitration, reduction and coupling to obtain isofetamid.

The progress of the reaction can be monitored using any suitable method, which can include, for example, chromatographic methods such as, e.g., high performance liquid chromatography (HPLC), thin layer chromatography (TLC), Gas chromatography (GC) and the like.

In yet another embodiment, the compound of formula (I) can be isolated from the reaction mixture by any conventional techniques well-known in the art. Such isolation techniques can be selected, without limitation, from the group consisting of concentration, extraction, precipitation, cooling, filtration, crystallization, centrifugation, and a combination thereof, followed by drying.

In yet another embodiment, the compound of formula (I) can be optionally purified by any conventional techniques well-known in the art. Such purification techniques can be selected, without limitation, from the group consisting of precipitation, crystallization, extraction, slurrying, washing in a suitable solvent, filtration through a packed-bed column, dissolution in an appropriate solvent, reprecipitation by addition of a second solvent in which the compound is insoluble, and a combination thereof.

In a further aspect, the present disclosure provides a process for preparation of isofetamid comprising:
ai) preparing compound of formula (V) according to the process as described in the second aspect;
bi) providing reaction conditions for preparation of isofetamid.

According to an embodiment the reaction conditions in step bi) include but are not limited to hydrolysis, nitration, reduction and coupling to obtain isofetamid.

The progress of the reaction can be monitored using any suitable method, which can include, for example, chromatographic methods such as, e.g., high performance liquid chromatography (HPLC), thin layer chromatography (TLC), Gas chromatography (GC), and the like.

In yet another embodiment, the compound of formula (V) can be isolated from the reaction mixture by any conventional techniques well-known in the art. Such isolation techniques can be selected, without limitation, from the group consisting of concentration, extraction, precipitation, cooling, filtration, crystallization, centrifugation, and a combination thereof, followed by drying.

In yet another embodiment, the compound of formula (V) can be optionally purified by any conventional techniques well-known in the art. Such purification techniques can be selected, without limitation, from the group consisting of precipitation, crystallization, extraction, slurrying, washing in a suitable solvent, filtration through a packed-bed column, dissolution in an appropriate solvent, reprecipitation by addition of a second solvent in which the compound is insoluble, and a combination thereof.

In a further aspect of the present disclosure provide a process for preparation of isofetamid comprising:
aj) preparing compound of formula (X) according to the process as described in the fourth aspect;
bj) providing reaction conditions for preparation of isofetamid.

According to an embodiment the reaction conditions in step bj) include but are not limited to coupling to obtain isofetamid.

The progress of the reaction can be monitored using any suitable method, which can include, for example, chromatographic methods such as, e.g., high performance liquid chromatography (HPLC), thin layer chromatography (TLC), Gas chromatography (GC), and the like.

In yet another embodiment, the compound of formula (X) can be isolated from the reaction mixture by any conventional techniques well-known in the art. Such isolation techniques can be selected, without limitation, from the group consisting of concentration, extraction, precipitation, cooling, filtration, crystallization, centrifugation, and a combination thereof, followed by drying.

In yet another embodiment, the compound of formula (X) can be optionally purified by any conventional techniques well-known in the art. Such purification techniques can be selected, without limitation, from the group consisting of precipitation, crystallization, extraction, slurrying, washing in a suitable solvent, filtration through a packed-bed column, dissolution in an appropriate solvent, reprecipitation by addition of a second solvent in which the compound is insoluble, and a combination thereof.

In the present disclosure, unless otherwise indicated, all the specific descriptions made in the first, second, third and fourth aspects apply to the fifth and other aspect as all relevant descriptions have been copied here. For example, unless otherwise indicated, all the specific descriptions on X, R¹, R², and R³ specified in the first aspect and second aspect apply to the fourth aspect as all relevant specific descriptions have been copied here; all the specific descriptions on steps a), b), c), d), e), a1), b1), and relevant materials used therein (e.g., bases, solvents, etc.), conditions (e.g., temperature and time, etc.) and the like specified in the second aspect apply to the fourth aspect as all relevant specific descriptions have been copied here; all the specific descriptions on steps B), c), and relevant materials used therein (e.g., bases, solvents, etc.), conditions (e.g., temperature and time, etc.) and the like specified in third aspect apply to the fourth aspect as all relevant specific descriptions have been copied here; and all the specific descriptions on steps i), ii) and iii) and relevant materials used therein (e.g., bases, solvents, etc.), conditions (e.g., temperature and time, etc.) and the like specified in the fourth aspect apply to the fifth and other aspect as all relevant specific descriptions have been copied in the corresponding places in the fifth and other aspects.

In some embodiments of the present disclosure, the processes for producing a compound of Formula (I) and intermediates of the general Formulas (II), (V) and (X), provide increased synthetic yields, as well as increasing operational simplicity in terms of telescopic process or even one-pot process.

Each embodiment disclosed herein is contemplated as being applicable to each of the other disclosed embodiments. Thus, all combinations of the various elements described herein are within the scope of the invention. In addition, the elements recited in process embodiments can be used in combination with compound embodiments described herein and vice versa.

This invention will be better understood by reference to the Examples which follow, but those skilled in the art will readily appreciate that the specific experiments detailed are only illustrative of the invention as described more fully in the claims which follow thereafter.

The invention is illustrated by the following examples without limiting it thereby.

### EXAMPLES

### Example 1

An exemplary experimental procedure for 1-(4-isopropoxy-2-methylphenyl)-2-methylpropan-1-one of formula (I) is described as follows:

### Step 1: Preparation of Grignard Reagent (Compound (III) X=Br, R¹=methyl, R²=isopropyl)

46 g THF, 26 g Mg and 5 g of 3 M solution of MeMgCl as initiator under N₂ were added to a flask at room temperature. The mixture was heated to 35° C. and 5 g of 1-bromo-4-isopropoxy-2-methylbenzene (compound II) from Example 2 was added to the flask at the same temperature. The mixture was heated at this temperature about half an hour up to the process initiation. At this moment reaction temperature rise to about 55° C. The mixture was cooled to 50° C. and then 247 g of 1-bromo-4-isopropoxy-2-methylbenzene in the mixture of 110 g THF and 440 g of toluene was added dropwise during about 6 hours keeping the temperature in the field 50-60° C. After the end of feeding the reaction mass was kept at the same conditions about 1 additional hour up to the reduction of starting material lower than 0.5 area % according to GC analysis.

### Step 2: Preparation of Compound (V) R¹=methyl, R²=isopropyl, R³=isopropyl

Reaction mixture from Step 1 was cooled to room temperature and 73 g of isobutyronitrile were added dropwise during 6 hours to flask at 25-35° C. After the feeding the reaction mass was kept at the same conditions for an hour to complete the reaction.

¹H NMR (Bruker, 400 MHz, MeOD) for Compound (V) R¹=methyl, R²=isopropyl, R³=isopropyl: 7.10 (d, 1H), 6.80 (s, 1H), 6.78 (d, 1H), 4.61 (m, 1H), 3.27 (s, 3H), 2.90 (m, 1H), 1.32 (s, 6H), 1.16 (d, 6H).

### Step 3: Preparation of Compound (I) R¹=methyl, R²=isopropyl, R³=isopropyl

To the separated flask 490 g of 15% by weight HCl were introduced at room temperature and the reaction mixture from Step 2 was added dropwise within 3 hours under intensive stirring and cooling the mixture below 50° C. After the feeding reaction mass was stirred at 50-55° C. about 5 hours up to the reduction of compound (V) concentration below 0.5 area % according to the HPLC analysis. Reaction mass was cooled to 40° C., stirring was stopped and phases were separated. Upper organic phase was washed with 50 g of water at the same temperature and the solvent was distilled out under the vacuum 100 mbar at the temperature about 100° C. The residue was distilled under vacuum 3 mbar at 150° C. thereby obtaining 1-(4-isopropoxy-2-methylphenyl)-2-methylpropan-1-one as a light yellow liquid.

Yield of 1-(4-isopropoxy-2-methylphenyl)-2-methylpropan-1-one: 229 g (75% based on compound (II)).

### Example 2

An exemplary experimental procedure for 1-bromo-4-isopropoxy-2-methylbenzene of formula (II) is described as follows:

### Step 1: Preparation of Compound (VII) R¹=methyl, R²=isopropyl

737.3 g ethanol, 491.5 g m-cresol (Compound VI) and 350.7 g KOH were added to a flask at the room temperature and heated to 75° C. 698.8 g 2-bromopropane was then added dropwise at about 75° C. An additional 17.5 g KOH and 34.9 g 2-bromopropane were then added to the flask at the same temperature. The reaction was lasted for about 3 hours at the same temperature until the remaining m-cresol concentration in the reaction mixture is reduced below 0.5% by GC area.

### Step 2: Preparation of Compound (II) X=Br, R¹=methyl, R²=isopropyl

In the same vessel the mixture comprising Compound VII and excess of potassium bromide salt was then cooled to 5-10° C. and 551.7 g 50% aq. H₂SO₄, and 535.7 g 30% aq. H₂O₂ were then added dropwise during 5 hours while maintaining the temperature at 5-15° C. The reaction was lasted for additional 8 hours at 15-25° C. until the remaining 1-isopropoxy-3-methylbenzene (Compound VII) concentration was reduced below 0.5% by GC area. The resulting mixture was filtered at room temperature in order to remove the resulting potassium sulfate salt which was rinsed with 245.8 g of ethanol. The resulting two-phase filtrate was separated, and the oil phase was washed with 147.5 g H₂O. The obtained oil phase was distilled under vacuum to collect the desired product, 1-bromo-4-isopropoxy-2-methylbenzene with assay 91% and yield 85%.

### Example 3

An exemplary experimental procedure for 2-amino-1-(4-isopropoxy-2-methylphenyl)-2-methylpropan-1-one of formula (X) is described as follows:

### Step 1: Preparation of 2-bromo-1-(4-isopropoxy-2-methylphenyl)-2-methylpropan-1-one Compound (VIII)

484.2 g 1-(4-isopropoxy-2-methylphenyl)-2-methylpropan-1-one (Compound I), 236.8 g DMSO and 182.0 g Acetic acid were added to a flask and heated to 75° C. At this temperature 505.7 g 48% aq. HBr was added dropwise during 3 hours. The reaction was lasted for 8 additional hours at 70-75° C. until the remaining concentration of Compound I was reduced below 1.0% by GC area. The mixture was cooled to 40° C. and then 881.2 g toluene and 150 g of water were added. After phase separation, the organic phase was cooled to 25° C. and 226.7 g 30% aq. H₂O₂ was then added dropwise at 25-30° C. The resulting mixture was phase separated and the organic phase was washed with 132.2 g H₂O.

After toluene distillation 661 g of 2-bromo-1-(4-isopropoxy-2-methylphenyl)-2-methylpropan-1-one were separated.

### Step 2: Preparation of 1-(4-isopropoxy-2-methylphenyl)-2-methyl-2-nitropropan-1-one (IX)

1137.0 g DMSO, 198.6 g NaNO₂ and 6.2 g TBAB were added to a flask at room temperature and the resulting product of Step 1 was fed stepwise at 28-32° C. for 6 hours. The reaction was lasted for about 5 additional hours at the same temperature until the remaining concentration of Compound VIII was reduced below 0.5% by GC area. After completion of the reaction, 682.2 g ethyl acetate was added into the reaction mixture at room temperature and the mixture was filtered to remove the resulted sodium salt which was rinsed with 454.8 g ethyl acetate. The resulting filtrate was washed two times with 100 g of H₂O.

Ethyl acetate solution of 1-(4-isopropoxy-2-methylphenyl)-2-methyl-2-nitropropan-1-one contains about 33% (474 g) of the product.

### Step 3: Preparation of 2-amino-1-(4-isopropoxy-2-methylphenyl)-2-methylpropan-1-one (X)

810 g of iron powder, 2570 g H₂O and 325 g of ethyl acetate were added to a flask and heated to 40-45° C. At this temperature the resulting 1-(4-isopropoxy-2-methylphenyl)-2-methyl-2-nitropropan-1-one from Step 2 was added dropwise to the mixture during 8 hours. The reaction was lasted for about 12 additional hours at the same conditions until concentrations of Compound IX was below 0.5% by HPLC area. The mixture was then filtered at room temperature to separate the Fe mud from the reaction solution. The resulting filtrate was phase separated. The upper organic phase was concentrated under vacuum. After concentration, an oil containing the desired 2-amino-1-(4-isopropoxy-2-methylphenyl)-2-methylpropan-1-one (X) was obtained.

Yield of 2-amino-1-(4-isopropoxy-2-methylphenyl)-2-methylpropan-1-one (X): 380 g (90%).

### Example 4

An exemplary experimental procedure for 4-isopropoxy-2-methylbenzonitrile of formula (XIII) is described as follows:

To a 500 mL four-necked flask were fed 124.0 g of DMSO, 124.0 g of toluene, 123.9 g of 2-bromo-5-isopropoxytoluene (Compound II), 20.8 g of NaBr and 60.0 g of CuCN. The mixture was heated and the traces of water were distilled out by azeotropic distillation at 130-140° C. Rest of the toluene was distilled out under vacuum minus 0.09 Mpa at the temperature 90-110° C. The mixture was heated to 145-150° C. and stirred at this temperature for 15 hours until the remaining concentration of Compound II was less than 4% by GC area.

The mixture of DMSO and Compound XIII was distilled under vacuum 500 Pa at the temperature 90-120° C. To the distillate 124.0 g of toluene and 124.0 g of H₂O were added and mixture was stirred for 0.5 hour. The phases were separated at ambient temperature. The organic phase was washed with 124.0 g of H₂O and dried by azeotropic distillation. Dry solution of 4-isopropoxy-2-methylbenzonitrile of formula (XIII) in toluene may be used on the next step without any additional purification.

Otherwise the reaction mixture was concentrated under vacuum minus 0.09 Mpa at the temperature up to 110° C. 61.0 g of 4-isopropoxy-2-methylbenzonitrile of formula (XIII) was obtained as a light-yellow oil with purity 90%.

### Example

An exemplary experimental procedure for 1-(4-isopropoxy-2-methylphenyl)-2-methylpropan-1-one of formula (I) is described as follows:

0.7 mol of commercially available isopropyl magnesium bromide of formula (XII) in 186 g of THF was introduced to a 500 mL four-necked flask and 57.7 g, 0.5 mol of 4-isopropoxy-2-methylbenzonitrile of formula (XIII) in 58.0 g toluene was fed to the solution. Reaction mixture was heated to the temperature 60-65° C. and stirred at this temperature for 18 hours. When the reaction was completed (synthesis of 1-(4-isopropoxy-2-methylphenyl)-1-imino-2-methylpropan—Compound V, ¹H NMR data are the same as in Example 1), 450.0 g of 15% hydrochloric acid were added into the mixture within 1 hour at the temperature below 50° C. The resulting mixture was held for 2 hours at 50-55° C., cooled to the temperature 30-40° C. and the phases were separated. Upper organic phase was washed with 40 g of water and concentrated under vacuum minus 0.09 Mpa at the temperature up to 110° C. to obtain 75.2 g of 1-(4-isopropoxy-2-methylphenyl)-2-methylpropan-1-one of formula (I) as a light-yellow oil with purity about 85%.

All publications, patents and patent applications mentioned in this specification are herein incorporated in their entirety by reference into the specification, to the same extent as if each individual publication, patent or patent application was specifically and individually indicated to be incorporated herein by reference.

The above examples illustrate the practice of the present subject matter in some of its embodiments but should not be construed as limiting the scope of the present subject matter. Other embodiments apparent to persons of ordinary skill in the art from consideration of the specification and examples herein that fall within the spirit and scope of the appended claims are part of this invention. The specification, including the examples, is intended to be exemplary only, without limiting the scope and spirit of the invention.

What is claimed is:

1. A compound of Formula (V) 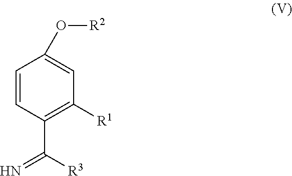
wherein:
R¹ is H, a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl;
R² is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and
R³ is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl.

2. The compound of claim 1, wherein R¹ is H, a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R² is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R³ is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl.

3. The compound of claim 1, wherein R¹ is H, a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R² is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R³ is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl.

4. The compound of claim 1, wherein R¹ is H, a C₁-C₃ alkyl, or a C₃-C₆ cycloalkyl, in which the alkyl may be substituted with a halogen, and the cycloalkyl may be substituted with a halogen, a C₁-C₆ alkyl or haloalkyl; R² is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl; wherein R³ is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl.

5. The compound of claim 1, wherein R¹ is H or a C₁-C₃ alkyl, in which the alkyl may be substituted with a halogen; R² is a C₁-C₄ straight or C₃-C₄ branched alkyl, in which the alkyl may be substituted with a halogen; R³ is a C₁-C₄ straight or C₃-C₄ branched alkyl, in which the alkyl may be substituted with a halogen.

6. The compound of claim 1, wherein R¹ is C₁-C₃ straight alkyl, in which the alkyl may be substituted with a halogen; R² is a C₃-C₄ branched alkyl, in which the alkyl may be substituted with a halogen; R³ is a C₃-C₄ branched alkyl, in which the alkyl may be substituted with a halogen.

7. The compound of claim 1, wherein R¹ is methyl; R² is isopropyl; and R³ is isopropyl.

8. A process for preparing a compound of Formula (V) according to any one of claims 1 to 7, 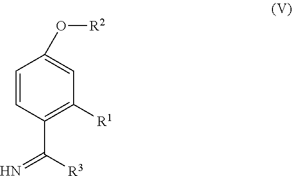
comprising:
b) reacting a compound of Formula (III) with a compound of Formula (IV) 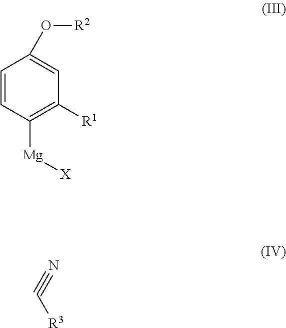
or
b1) reacting a compound of Formula (XII) with a compound of Formula (XIII) 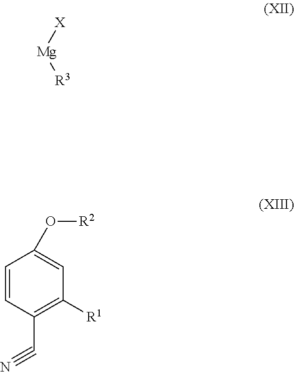
to prepare a compound of Formula (V);
wherein:
X is a halogen;
R¹, R², and R³ are defined as any one of claims 1 to 7.

9. The process of claim 8, wherein when the process comprises a step b), the process further comprises a step a) reacting the compound of Formula (II) with magnesium to prepare the compound of Formula (III) 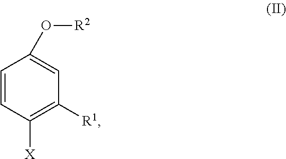
wherein: X, R¹, and R² are defined as in claim 8.

10. The process of claim 8, wherein when the process comprises a step b1), the process further comprises a step a1) reacting the compound of Formula (XI) with magnesium to prepare the compound of Formula (XII) 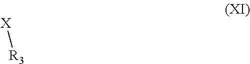
wherein: X and R³ are defined as in claim 8.

11. The process of claim 9, wherein steps a) and b) are carried out sequentially as a telescopic process.

12. The process of claim 10, wherein steps a1) and b1) are carried out sequentially as a telescopic process.

13. The process of any one of claims 9 and 11, wherein the process further comprises:
f) reacting the compound of Formula (VI) with an alkylating agent 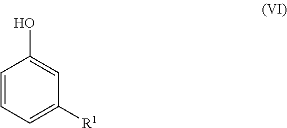
to prepare a compound of Formula (VII) 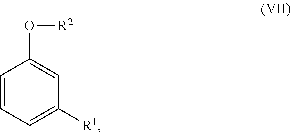
g) halogenating the compound of Formula (VII) with a halogenating agent to prepare the compound of Formula (II), 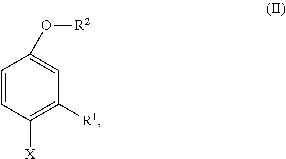
wherein, X, R¹ and R² are defined as in claim 9.

14. The process of claim 13, wherein step d) is carried out with an alkyl halide and in the presence of a base.

15. The process of claim 13, wherein step e) and step d) are carried out sequentially as a telescopic process.

16. The process of claim 14, wherein the base is selected from the group consisting of alkali metal hydroxides, alkali metal carbonates, hydrides, alkaline earth metal hydroxides and alkaline earth metal carbonates.

17. The process of claim 13, wherein the halogenating agent in step e) is a chlorinating agent selected from the group consisting of NCS, Cl₂, dichlorodimethyl hydantoin, trichloroisocyanuric acid, N-chlorophthalimide, sulfuryl chloride and the mixtures thereof.

18. The process of claim 13, wherein the halogenating agent in step e) is a brominating agent selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid, bromodimethylsulfonium bromide, 5,5-dibromomeldrum's acid, bis(2,4,6-trimethylpyridine)-bromonium hexafluorophosphate, bromine monochloride and the mixtures thereof

19. The process of any one of claims 13-18, wherein the halogenating in step e) is carried out by oxyhalogenating process using an oxidizing agent and source of halogen ions under acidic conditions.

20. The process of claim 19, wherein the oxidizing agent is selected from the group consisting of hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, oxone, DMSO, and mixtures thereof.

21. The process of any one of claims 19-20, wherein the source of halogen ions is a hydrogen halide or a mixture of a strong acid and an alkali metal or alkaline earth metal salt of a hydrogen halide.

22. The process of any one of claims 13-21, wherein a phase transfer catalyst is used in step d) and/or step e).

23. The process of any one of claims 8-22, wherein X is Cl or Br.

24. A process for preparing a compound of Formula (I), 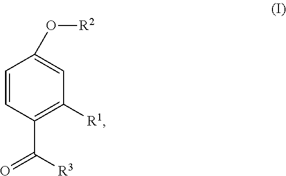
comprising:
B) preparing the compound of Formula (V) according to the process of any one of claims 8 to 23; 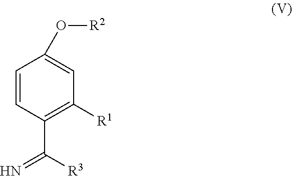
c) hydrolyzing the resulting compound of Formula (V);
wherein: R¹, R², and R³ are defined as any one of claims 8 to 23.

25. The process of claim 24, wherein the hydrolysis in step c) is acid-catalyzed hydrolysis or base-catalyzed hydrolysis.

26. The process of any one of claims 24-25, wherein when the process comprises steps a), b) and c), steps a) and b), steps b) and c), or steps a), b) and c) are carried out sequentially as a telescopic process.

27. The process of any one of claims 24-25, wherein when the process comprises steps a1), b1) and c), steps a1) and b1), steps b1) and c), or steps a1), b1) and c) are carried out sequentially as a telescopic process.

28. A process for preparing a compound of Formula (X) comprises:
i) halogenating a compound of Formula (I) with a halogenating agent to prepare a compound of Formula (VIII) 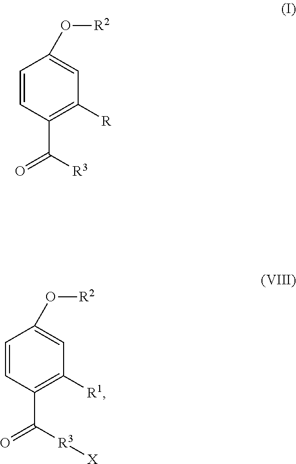
ii) substitution in the compound of Formula (VIII) to prepare a compound of Formula (IX) 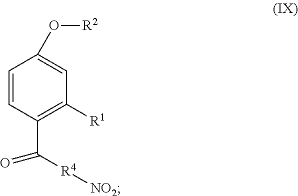
iii) reducing the compound of Formula (IX) to prepare a compound of Formula (X) 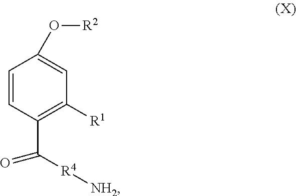
wherein:
X is a halogen;
R¹ is H, a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl;
R² is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and
R³ is a C₁-C₁₀ straight or C₃-C₁₀ branched alkyl, a C₃-C₁₀ cycloalkyl, a C₇-C₁₀ aralkyl, or a C₆-C₁₀ aryl, in which the alkyl may be substituted with a halogen, and the cycloalkyl, aralkyl and aryl may be substituted with a halogen, a C₁-C₁₀ alkyl or haloalkyl, or a C₁-C₁₀ alkoxyl or haloalkoxyl; and
R⁴ is a C₁-C₁₀ straight or C₃-C₁₀ branched alkanediyl, a C₃-C₁₀ cycloalkylene, a C₇-C₁₀ aralkylene, or a C₆-C₁₀ arylene, in which the alkanediyl may be substituted with a halogen, and the cycloalkylene, aralkylene and arylene may be substituted with a halogen, a C₁-C₁₀ alkyl or haloalkyl, or a C₁-C₁₀ alkoxyl or haloalkoxyl.

29. The process of claim 28, wherein X is F, Cl or Br; R¹ is H, a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R² is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; R³ is a C₁-C₆ straight or C₃-C₆ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₁₀ alkyl or haloalkyl, or a C₁-C₁₀ alkoxyl or haloalkoxyl; and R⁴ is a C₁-C₆ straight or C₃-C₆ branched alkanediyl, a C₃-C₆ cycloalkylene, or a C₇-C₁₀ aralkylene, in which the alkanediyl may be substituted with a halogen, and the cycloalkylene and aralkylene may be substituted with a halogen, a C₁-C₁₀ alkyl or haloalkyl, or a C₁-C₁₀ alkoxyl or haloalkoxyl.

30. The process of claim 28, wherein X is F, Cl or Br; R¹ is H, a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R² is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; R³ is a C₁-C₄ straight or C₃-C₄ branched alkyl, a C₃-C₆ cycloalkyl, or a C₇-C₁₀ aralkyl, in which the alkyl may be substituted with a halogen, and the cycloalkyl and aralkyl may be substituted with a halogen, a C₁-C₆ alkyl or haloalkyl, or a C₁-C₆ alkoxyl or haloalkoxyl; and R⁴ is a C₁-C₄ straight or C₃-C₄ branched alkanediyl, a C₃-C₆ cycloalkylene, or a C₇-C₁₀ aralkylene, in which the alkanediyl may be substituted with a halogen, and the cycloalkylene and aralkylene may be substituted with a halogen, a C₁-C₆ alkyl or haloalkyl, or a C₁-C₆ alkoxyl or haloalkoxyl.

31. The process of claim 28, wherein X is F, Cl or Br; R¹ is H, a C₁-C₃ alkyl, or a C₃-C₆ cycloalkyl, in which the alkyl may be substituted with a halogen, and the cycloalkyl may be substituted with a halogen, a C₁-C₆ alkyl or haloalkyl; R² is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl; wherein R³ is a C₁-C₄ straight or C₃-C₄ branched alkyl, or a C₃-C₆ cycloalkyl, in which the alkyl may be substituted with a halogen, and the cycloalkyl may be substituted with a halogen, a C₁-C₆ alkyl or haloalkyl; and R⁴ is a C₁-C₄ straight or C₃-C₄ branched alkanediyl, or a C₃-C₆ cycloalkylene, in which the alkanediyl may be substituted with a halogen, and the cycloalkylene may be substituted with a halogen, a C₁-C₆ alkyl or haloalkyl.

32. The process of claim 28, wherein X is F, Cl or Br; R¹ is H or a C₁-C₃ alkyl, in which the alkyl may be substituted with a halogen; R² is a C₁-C₄ straight or C₃-C₄ branched alkyl, in which the alkyl may be substituted with a halogen; R³ is a C₁-C₄ straight or C₃-C₄ branched alkyl, in which the alkyl may be substituted with a halogen; and R⁴ is a C₁-C₄ straight or C₃-C₄ branched alkanediyl, in which the alkanediyl may be substituted with a halogen.

33. The process of claim 28, wherein X is F, Cl or Br; R¹ is C₁-C₃ straight alkyl, in which the alkyl may be substituted with a halogen; R² is a C₃-C₄ branched alkyl, in which the alkyl may be substituted with a halogen; R³ is a C₃-C₄ branched alkyl, in which the alkyl may be substituted with a halogen; and R⁴ is a C₃-C₄ branched alkanediyl, in which the alkanediyl may be substituted with a halogen.

34. The process of claim 28, wherein X is Br; R¹ is methyl; R² is isopropyl; R³ is isopropyl, and R⁴ is 2,2-propandiyl.

35. The process of claim 28, wherein the halogenating agent in step i) is a chlorinating agent selected from the group consisting of NCS, Cl₂, dichlorodimethyl hydantoin, trichloroisocyanuric acid, N-chlorophthalimide, sulfuryl chloride and the mixtures thereof.

36. The process of claim 28, wherein the halogenating agent in step i) is a brominating agent selected from the group consisting of NBS, Br₂, dibromodimethyl hydantoin, tribromoisocyanuric acid, N-bromophthalimide, N-bromosaccharin, monosodium bromoisocyanurate hydrate, dibromoisocyanuric acid, bromodimethylsulfonium bromide, 5,5-dibromomeldrum's acid, bis(2,4,6-trimethylpyridine)-bromonium hexafluorophosphate, bromine monochloride and the mixtures thereof

37. The process of any one of claims 28-36, wherein the halogenating in step i) is carried out by oxyhalogenating process using an oxidizing agent and source of halogen ions under acidic conditions.

38. The process of claim 37, wherein the oxidizing agent is selected from the group consisting of hydrogen peroxide, benzoyl peroxide, tert-butyl peroxide, m-chloroperoxybenzoic acid, peroxyacetic acid, peroxybenzoic acid, magnesium monoperoxyphthalate, potassium peroxymonosulfate, oxone, DMSO, and mixtures thereof.

39. The process of any one of claims 37-38, wherein the source of halogen ions is a hydrogen halide or a mixture of a strong acid and an alkali metal or alkaline earth metal salt of a hydrogen halide.

40. The process of any one of claims 28-39, wherein a phase transfer catalyst is used in step ii).

41. The process of any one of claims 28-40, wherein a nitrite salt is used in step ii).

42. The process of claim 41, wherein the nitrite salt is selected from the group consisting of alkali metal nitrite salt and alkali earth metal nitrite salt.

43. The process of any one of claims 28-42, wherein steps i) and ii), steps ii) and iii), or steps i), ii), and iii) are carried out sequentially as a telescopic process.

44. The process of any one of claims 28-43, wherein the compound of Formula (I) is prepared according to the process of any one of claims 24-27.

45. Use of the compound of formula (V) as prepared according to any one of claims 8-22 for preparing isofetamid.

46. Use of the compound of formula (I) as prepared according to any one of claims 23-27 for preparing isofetamid.

47. Use of the compound of formula (X) as prepared according to any one of claims 28-44 for preparing isofetamid.

48. A process for preparation of isofetamid comprising: aa) preparing a compound of formula (I) according to the process of any one of claims 23-27; bb) preparing isofetamid from the compound of formula (I).

49. A process for preparation of isofetamid comprising: ai) preparing a compound of formula (V) according to the process of any one of claims 8-22; bi) preparing isofetamid from the compound of formula (V).

50. A process for preparation of isofetamid comprising: aj) preparing a compound of formula (X) according to the process of any one of claims 28-44; bj) preparing isofetamid from the compound of formula (X).
